(19) United States
(12) Patent Application Publication
(10) Pub. No.: US 2007/0060818 A1
(43) Pub. Date: Mar. 15, 2007

(54) Blood-vessel-endothelium-function inspecting apparatus

(75) Inventors: Katsushi Iikubo, Nagoya-shi (JP); Hidehito Sasaki, Nagoya-shi (JP); Hidenori Suzuki, Nagoya-shi (JP); Hiromasa Tsukahara, Nagoya-shi (JP); Chikao Harada, Nagoya-shi (JP); Hitoshi Hirano, Nagoya-shi (JP); Hiroshi Masuda, Nagoya-shi (JP)

Correspondence Address: OLIFF & BERRIDGE, PLC, P.O. BOX 19928, ALEXANDRIA, VA, 22320, US

(73) Assignee: UNEX CORPORATION, Nagoya-shi (JP)

(21) Appl. No.: 11/506,847

(22) Filed: Aug. 21, 2006

(30) Foreign Application Priority Data
Aug. 29, 2005 (JP) 2005-247948

(51) Int. Cl. A61B 8/00

(52) U.S. Cl. 600/438; 600/437

(57) ABSTRACT

An apparatus for inspecting a function of an endothelium of a blood vessel of a living being, by stopping, using an inflatable cuff wound around a forearm of the living being, a flow of blood in a blood vessel of the forearm, subsequently ending the stopping of the flow of blood, and causing an ultrasonic probe placed on a skin of an upper arm of the living being to emit, from an emission surface thereof, an ultrasonic wave toward a blood vessel under the skin, the apparatus including an elbow-portion support member which supports an elbow portion of the living being; and a hand-dorsal-portion support member which supports a dorsal portion of a hand of the living being and which cooperates with the elbow-portion support member to keep, in a space, the inflatable cuff wound around the forearm of the living being.

The present application is based on Japanese Patent Application No. 2005-247948 filed on Aug. 29, 2005, the contents of which are incorporated herein by reference.

### BACKGROUND OF THE INVENTION

1. Field of the Invention

The present invention relates to a blood-vessel-endothelium-function inspecting apparatus that inspects a function of an endothelium of a blood vessel of a living being, by causing an ultrasonic probe placed on a skin of a portion of the living being to emit, from an emission surface thereof, an ultrasonic wave toward the blood vessel under the skin.

2. Related Art Statement

A function of an endothelium of a blood vessel is represented by, e.g., a rate of increase of diameter of the blood vessel at the time of reactive hyperemia. Since the function of endothelium of blood vessel is closely related to degree of arteriosclerosis, Japanese Patent Application Publication No. 2003-245280, for example, has proposed a non-invasive and simple method of inspecting a function of an endothelium of a blood vessel so as to be able to make an early diagnosis of arteriosclerosis.

In the proposed method, an ultrasonic probe that is supported by a free end portion of a robot arm is pressed against an upper arm of a living subject, and an image of a blood vessel before flow of blood in the blood vessel is stopped and an image of the blood vessel after the stopping of the flow of blood is ended are obtained. Then, a rate of increase of a diameter, dmax, of the blood vessel at the time of dilation, from a diameter, d, of the same at the time of constriction, i.e., an FMD (flow-mediated dilation) value [=100×(dmax−d)/d] is calculated, and is used in evaluating a degree of arteriosclerosis.

### SUMMARY OF THE INVENTION

Meanwhile, in the above-indicated blood-vessel-endothelium-function inspecting apparatus, a cross-section shape of a blood vessel of a living being is measured by causing an ultrasonic probe placed on, e.g., a skin of a portion of the living being to emit, from an emission surface thereof, an ultrasonic wave toward the blood vessel under the skin, for example, after stopping of a flow of blood by a cuff is ended or after flow of blood is changed by use of a drug, or heating. However, when flow of blood is stopped by a cuff wound around the portion of the living being, e.g., a forearm, the skin of that portion may be drawn by the inflation of the cuff, or the forearm may be moved by the inflation. That is, the ultrasonic probe that is supported by a sensor holding apparatus such as a robot arm may be moved relative to an artery, such as a brachial artery, of another portion adjacent to the portion to which the probe is applied. This leads to distorting the image of the blood vessel and raising such a problem that an accurate diameter of the blood vessel cannot be measured.

It is therefore an object of the present invention to provide a blood-vessel-endothelium-function inspecting apparatus that can measure an accurate diameter of a blood vessel of a living being irrespective of whether a cuff wound around a portion of the living being may be inflated.

The above object has been achieved by the present invention. According to a first mode of the present invention, there is provided an apparatus for inspecting a function of an endothelium of a blood vessel of a living being, by stopping, using an inflatable cuff wound around a forearm of the living being, a flow of blood in a blood vessel of the forearm, subsequently ending the stopping of the flow of blood, and causing an ultrasonic probe placed on a skin of an upper arm of the living being to emit, from an emission surface thereof, an ultrasonic wave toward a blood vessel under the skin, the apparatus comprising an elbow-portion support member which supports an elbow portion of the living being; and a hand-dorsal-portion support member which supports a dorsal portion of a hand of the living being and which cooperates with the elbow-portion support member to keep, in a space, the inflatable cuff wound around the forearm of the living being.

In the blood-vessel-endothelium-function inspecting apparatus in accordance with the first mode of the present invention, the elbow-portion support member supports the elbow portion of the living being, and the hand-dorsal-portion support member supports the dorsal portion of the hand of the living being and cooperates with the elbow-portion support member to keep, in the space, the inflatable cuff wound around the forearm of the living being. Therefore, the movement of the forearm caused by the inflation of the cuff can be effectively restrained. Thus, a relative movement of (a) the ultrasonic probe supported by a sensor holding apparatus and (b) a brachial artery to which the probe is applied can be prevented from being caused by the movement of the forearm that is related to the inflation of the cuff. Consequently a stable image of the blood vessel can be obtained and accordingly an accurate diameter of the blood vessel can be measured.

According to a second mode of the present invention, at least one of the elbow-portion support member and the hand-dorsal-portion support member is provided on a horizontal support surface of a support table such that the at least one of the elbow-portion support member and the hand-dorsal-portion support member is movable in a horizontal direction on the horizontal support surface.

In the blood-vessel-endothelium-function inspecting apparatus in accordance with the second mode of the present invention, at least one of the elbow-portion support member and the hand-dorsal-portion support member is provided on the horizontal support surface of the support table such that the at least one member is movable in the horizontal direction or directions on the horizontal support surface. Therefore, a position(s) of the elbow-portion support member and/or the hand-dorsal-portion support member can be adjusted to an optimum position(s) corresponding to the specific form of the body of the living being (or living subject) lying on, e.g., a bed, so that the arm of the living being can take a natural or relaxed posture.

According to a third mode of the present invention, the blood-vessel-endothelium-function inspecting apparatus further comprises a height adjusting device which adjusts a height of a support surface of at least one of the elbow-portion support member and the hand-dorsal-portion support member that supports a corresponding one of the elbow portion, and the dorsal portion of the hand, of the living being.

In the blood-vessel-endothelium-function inspecting apparatus in accordance with the third mode of the present invention, the height adjusting device adjusts the height of the support surface of at least one of the elbow-portion support member and the hand-dorsal-portion support member. Therefore, a position(s) of the elbow-portion support member and/or the hand-dorsal-portion support member can be adjusted to an optimum position(s) corresponding to the specific form of the body of the living being or subject lying on, e.g., a bed, so that the arm of the living subject can take a natural or relaxed posture.

According to a fourth mode of the present invention, the blood-vessel-endothelium-function inspecting apparatus further comprises a belt which is attached to the hand-dorsal-portion support member and which is to be grasped by the hand of the living being when the dorsal portion of the hand is supported by the hand-dorsal-portion support member.

In the blood-vessel-endothelium-function inspecting apparatus in accordance with the fourth mode of the present invention, the belt is attached to the hand-dorsal-portion support member, so that the belt may be grasped by the hand of the living being when the dorsal portion of the hand is supported by the hand-dorsal-portion support member. Therefore, during the inspection or measurement, the hand and arm of the living being can be kept stable, and accordingly a stable image of the blood vessel can be obtained. Based on the thus obtained stable image of the blood vessel, a diameter of the blood vessel can be measured with an increased accuracy.

According to a fifth mode of the present invention, there is provided an apparatus for inspecting a function of an endothelium of a blood vessel of a living being, by causing an ultrasonic probe placed on a skin of a portion of a living being to emit, from an emission surface thereof, an ultrasonic wave toward a blood vessel under the skin, the apparatus comprising a holding surface which is provided in a vicinity of the emission surface of the ultrasonic probe such that the holding surface is opposed to the skin, so that a coupling agent is interposed between the emission surface and the skin irrespective of whether the skin may be moved during the inspection.

In the blood-vessel-endothelium-function inspecting apparatus in accordance with the fifth mode of the present invention, the holding surface is provided in the vicinity of the emission surface of the ultrasonic probe such that the holding surface is opposed to the skin, so that the coupling agent may be interposed between the emission surface and the skin irrespective of whether the skin may be moved during the inspection. Therefore, even if the skin may be drawn by the inflation of the cuff, the coupling agent, such as a jelly, can be interposed with stability between the emission surface and the skin. Thus, a stable image of the blood vessel can be obtained, and accordingly a diameter of the blood vessel can be measured with stability.

According to a sixth mode of the present invention, the ultrasonic probe includes an end portion which supports a flange portion projecting therefrom in a direction away from the emission surface, and the flange portion has the holding surface as a surface thereof that is opposed to the skin.

In the blood-vessel-endothelium-function inspecting apparatus in accordance with the sixth mode of the present invention, the flange portion projects from the end portion of the ultrasonic probe, in the direction away from the emission surface thereof, and the flange portion has the holding surface as the surface thereof that is opposed to the skin. Therefore, even if the skin may be drawn by the inflation of the cuff, the coupling agent can be interposed with stability between the emission surface and the skin. Thus, a stable image of the blood vessel can be obtained, and accordingly a diameter of the blood vessel can be measured with stability.

According to a seventh mode of the present invention, there is provided an apparatus for inspecting a function of an endothelium of a blood vessel of a living being, by causing an ultrasonic probe placed on a skin of a portion of a living being to emit, from an emission surface thereof, an ultrasonic wave toward a blood vessel under the skin, the apparatus comprising a cross-section-image producing device which produces, based on the reflected ultrasonic wave detected by the ultrasonic probe, a cross-section image of the blood vessel; a blood-vessel-position seeking device which seeks, in a cross-section image of the blood vessel produced after stopping of a flow of blood in a blood vessel is ended, the blood vessel within a pre-set range corresponding to a position of the blood vessel before the flow of blood is stopped; and a blood-vessel-diameter calculating device which calculates a diameter of the blood vessel sought by the blood-vessel-position seeking device.

In the blood-vessel-endothelium-function inspecting apparatus in accordance with the seventh mode of the present invention, the cross-section-image producing means or device produces, based on the reflected ultrasonic wave detected by the ultrasonic probe, the cross-section image of the blood vessel, the blood-vessel-position seeking means or device seeks, in the cross-section image of the blood vessel produced after the stopping of the flow of blood in the blood vessel is ended, the blood vessel within the pre-set range corresponding to the position of the blood vessel before the flow of blood is stopped, and the blood-vessel-diameter calculating means or device calculates the diameter of the blood vessel sought by the blood-vessel-position seeking device. Therefore, even if the image of the blood vessel may be changed, e.g., because the skin is drawn by the inflation of the cuff or because the forearm is moved, the blood-vessel-position seeking device seeks the blood vessel, and the blood-vessel-diameter calculating device calculates the diameter of the blood vessel. Thus, a diameter of the blood vessel can be measured with stability.

### BRIEF DESCRIPTION OF THE DRAWINGS

The above and optional objects, features, and advantages of the present invention will be better understood by reading the following detailed description of the preferred embodiments of the invention when considered in conjunction with the accompanying drawings, in which:

FIG. 1 is a schematic view of a construction of a blood-vessel-endothelium-function inspecting apparatus as a first embodiment of the present invention;

FIG. 2 is an enlarged view of an end portion of a sensor holding apparatus of the inspecting apparatus of FIG. 1, the end portion supporting a universal joint holding an ultrasonic array, and a stopper device to stop or inhibit the universal rotation of the joint;

FIG. 3 is a view showing a state in which an endothelium function of a blood vessel is inspected, using the ultrasonic array provided with a flange portion, before flow of blood in the blood vessel is stopped by inflation of a cuff;

FIG. 4 is a view showing another state in which the endothelium function of the blood vessel is inspected, using the ultrasonic array provided with the flange portion, when the flow of blood in the blood vessel is being stopped by the inflation of the cuff,

FIG. 5 is a view showing another state in which the endothelium function of the blood vessel is inspected, using the ultrasonic array provided with the flange portion, after the stopping of flow of blood in the blood vessel is ended by deflation of the cuff;

FIG. 6 is a view showing a state in which an endothelium function of a blood vessel is inspected, using an ultrasonic array without a flange portion, before flow of blood in the blood vessel is stopped by inflation of a cuff;

FIG. 7 is a view showing another state in which the endothelium function of the blood vessel is inspected, using the ultrasonic array without a flange portion, when the flow of blood in the blood vessel is being stopped by the inflation of the cuff;

FIG. 8 is a view showing another state in which the endothelium function of the blood vessel is inspected, using the ultrasonic array without a flange portion, after the stopping of flow of blood in the blood vessel is ended by deflation of the cuff;

FIG. 9 is a view showing respective positions of an elbow-portion support member and a hand-dorsal-portion support member that respectively support an elbow portion, and a dorsal portion of a hand, of a living subject lying on a bed, when an endothelium function of a blood vessel of the subject is inspected using the inspecting apparatus of FIG. 1;

FIG. 10 is a view for explaining a construction of the hand-dorsal-portion support member whose height is adjustable;

FIG. 11 is a view for explaining another blood-vessel-endothelium-function inspecting apparatus as a second embodiment of the present invention;

FIG. 12 is a view for explaining another blood-vessel-endothelium-function inspecting apparatus as a third embodiment of the present invention;

FIG. 13 is a view corresponding to FIG. 3, for explaining an end portion of another ultrasonic probe employed by another blood-vessel-endothelium-function inspecting apparatus as a fourth embodiment of the present invention;

FIG. 14 is a view corresponding to FIG. 3, for explaining an end portion of another ultrasonic probe employed by another blood-vessel-endothelium-function inspecting apparatus as a fifth embodiment of the present invention;

FIG. 15 is a flow chart representing relevant steps of an operation of an electronic control device of each of the blood-vessel-endothelium-function inspecting apparatuses; and

FIGS. 16A, 16B, 16C, 16D, 16E, 16F, and 16G are views showing respective transverse-cross-section views, i.e., short-axis images of a blood vessel that correspond to the respective steps of the flow chart of FIG. 15.

### DETAILED DESCRIPTION OF PREFERRED EMBODIMENTS

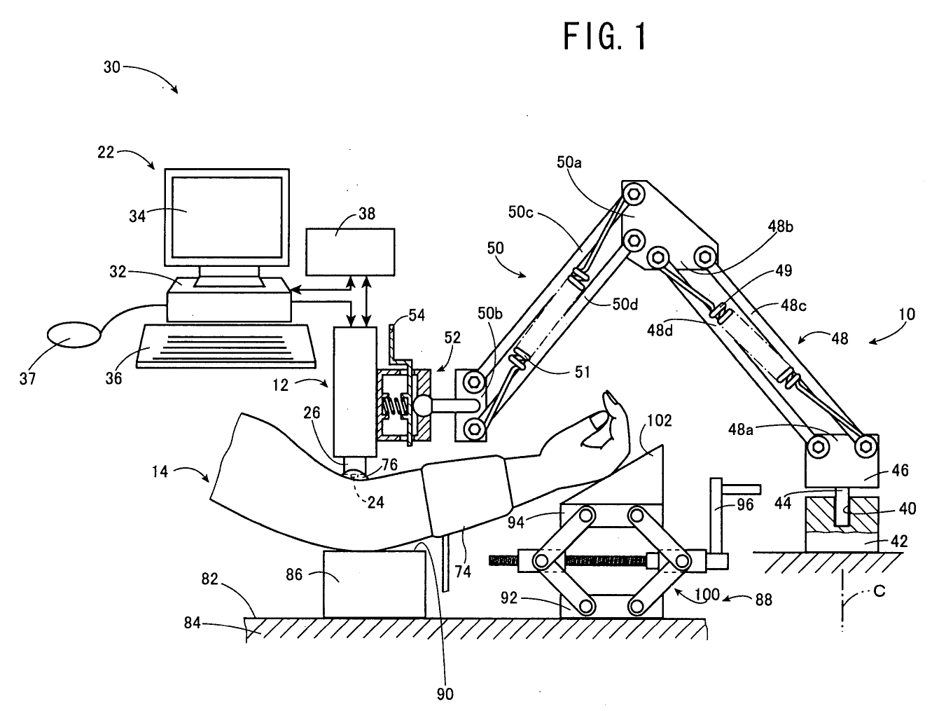

Hereinafter, there will be described preferred embodiments of the present invention in detail by reference to the drawings. FIG. 1 is a front view for explaining a blood-vessel-endothelium-function inspecting apparatus 30 including a blood-vessel-image measuring apparatus 22 which includes an ultrasonic probe 12 as a sensor, and a sensor holding apparatus 10 that holds the ultrasonic probe 12, and which measures, using the ultrasonic probe 12 held on a surface of a skin 18 of an upper arm 16 of a living being 14 (e.g., a living person) as an object to be inspected, a transverse-cross-section image (i.e., a short-axis image) and/or a longitudinal-cross-section image (i.e., a long-axis image) of a blood vessel (e.g., an artery) 20 located right below the skin 18.

The ultrasonic probe 12 functions as a sensor that detects physical information of a living being, and has a free-end portion 24 including a large number of ultrasonic transducers each of which is constituted by, e.g., a piezoelectric ceramics and which are arranged in one array (or two arrays parallel to each other); a multiple-axis driving or positioning device 26; and a main frame 28 that supports the free-end portion 24 via the multiple-axis positioning device 26.

The blood-vessel-endothelium-function inspecting apparatus 30 further includes an electronic control device 32 that is constituted by a so-called microcomputer; a monitor-image displaying device 34; a keyboard 36 and a mouse 37 as an input device; and an ultrasonic-wave control circuit 38. The electronic control device 32 controls the ultrasonic-wave control circuit 38 to supply drive signals to the ultrasonic array at the free-end portion 24 of the ultrasonic probe 12, so that the ultrasonic array emits ultrasonic waves, receive the ultrasonic waves reflected from the tissue located under the skin 18, and produce reflected-ultrasonic-wave signals. The control device 32 receives the reflected-ultrasonic-wave signals from the ultrasonic array, processes the thus received signals, produces an ultrasonic image of the tissue under the skin 18, and controls the monitor-image displaying device 34 to display the thus produced ultrasonic image. An outer surface of the free-end portion 24 corresponds to an emission surface, S, from which the ultrasonic array emits the ultrasonic waves. When the control device 32 produces the transverse-cross-section image (i.e., the short-axis image) of the blood vessel 20, the control device 32 controls the three-axis positioning device 26 to position the ultrasonic array of the free-end portion 24 relative to the blood vessel 20 such that the ultrasonic array extends in a direction perpendicular to the blood vessel 20; and when the control device 32 produces the longitudinal-cross-section image (i.e., the long-axis image) of the blood vessel 20, the control device 32 controls the three-axis positioning device 26 to position the ultrasonic array relative to the blood vessel 20 such that the ultrasonic array extends in a direction parallel to the blood vessel 20.

The ultrasonic-wave control circuit 38 carries out, according to a command supplied from the electronic control device 32, a beam-forming operation in which a predetermined number of transducers (e.g., 15 transducers) starting with one of opposite ends of the ultrasonic array are simultaneously driven such that each of the transducers generates an ultrasonic wave having a frequency of about 10 MHz with a predetermined phase difference from the phase of the ultrasonic wave generated by each of the two transducers located adjacent the each transducer on either side of the same. While the predetermined number of transducers are shifted one transducer by one in a direction from the above-indicated one end of the ultrasonic array to the other end thereof, the ultrasonic array sequentially generates, toward the blood vessel 20, respective ultrasonic beams each of which is convergent with respect to the direction of extension of the ultrasonic array. Each time the ultrasonic array generates the ultrasonic beam, it receives the ultrasonic beam reflected from the blood vessel 20, and inputs a signal representing the received, reflected ultrasonic beam, to the control device 32. An outer surface of the free-end portion 24 in which the ultrasonic array is provided is covered with an acoustic lens 92 that causes the ultrasonic beams to converge with respect to a direction perpendicular to the direction of extension of the ultrasonic array.

The electronic control device 32 synthesizes or produces, based on the reflected ultrasonic beams detected by the ultrasonic array, an ultrasonic transverse-cross-section image (i.e., short-axis image) of the blood vessel 20 located under the skin surface 18, and/or an ultrasonic longitudinal-cross-section image (i.e., long-axis image) of the blood vessel 20, and controls the monitor-image displaying device 34 to display the thus produced ultrasonic cross-section image(s) of the blood vessel 20. In addition, the control device 32 calculates, from the produced image(s) of the blood vessel 20, a diameter of the same 20, i.e., a diameter of an endothelium (i.e., an inner layer (tunica intima)) of the same 20. Moreover, for the purpose of evaluating a function of the endothelium of the blood vessel 20, the control device 32 calculates a rate of change (%) [=100×(dmax−d)/d, where d is a diameter of the endothelium (i.e., tunica intima) of the vessel 20 when the living being 14 is at rest; and dₘₐₓ is a maximum diameter of the endothelium of the vessel 20 after the flow of blood is resumed] of the diameter of the endothelium of the vessel 20 that represents FMD (flow-mediated dilation) following postischemia reactive hyperemia. Based on the calculated diameter of endothelium of the blood vessel 20, the control device 32 can calculate an accurate cross-section area of a lumen of the blood vessel 20 through which blood flows. In addition, the control device 32 can calculate an accurate flow rate of the blood based on a flow velocity of the blood detected by an ultrasonic Doppler device, not shown.

The ultrasonic probe 12 is held by the sensor holding apparatus 10, such that the probe 12 takes a desirable posture and touches, at a desirable or predetermined position in a three-dimensional space, the skin surface 18 of the upper arm 16 of the living being 14 as the object, without changing a shape of the blood vessel 20 located right below the skin surface 18. Usually, a well-known coupling agent such as a jelly is interposed between the skin surface 18 and the outer surface of the free end portion 24 of the ultrasonic probe 12, for the purpose of preventing the attenuation of ultrasonic waves, and/or the reflection or scattering thereof at the interface of the two elements 18, 24, and thereby obtaining clear ultrasonic images. The jelly may be gel of a hydrophilic polymer that contains water at a high rate and has an intrinsic impedance [=(sound speed)×(density)] sufficiently higher than that of air, and accordingly effectively restrains the attenuation of ultrasonic-waves signals transmitted and received. The jelly is, e.g., agar, but it may be replaced with a water bag, i.e., a water packed in a resin-based bag; olive oil; or glycerin.

The sensor holding apparatus 10 is fixed in position to a support member such as a desk or a seat. More specifically described, the sensor holding apparatus 10 includes a base member 42 having a fitting hole 40 extending along a vertical axis line, C; and a rotatable member 46 that has a fitting axis portion 44 that fits in the fitting hole 40 such that the axis portion 44 is rotatable relative thereto, so that the rotatable member 46 is rotatable about the vertical axis line C relative to the base member 42. The sensor holding apparatus 10 additionally includes a first link device 48 that is constituted by four links 48a, 48b, 48c, 48d including a horizontal, first stationary link 48a fixed to (i.e., integral with) the rotatable member 46; a second link device 50 that is constituted by four links 50a, 50b, 50c, 50d including a vertical, second stationary link 50a fixed to (i.e., integral with) an end portion of the first link device 48; a universal joint 52 that is fixed to an end portion of the second link device 50, connects the ultrasonic probe 12 to the same 50, and supports the probe 12 such that the probe 12 is universally rotatable; and a stopper device 56 that includes an operable lever 54 and that fixes the universal joint 52 while the lever 54 is not operated by an operator, and releases the fixation of the joint 52, i.e., permits the universal rotation of the joint 52 while the lever 54 is operated by the operator.

The first link device 48 includes the first stationary link 48a; a first movable link 48b extending parallel to the first stationary link 48a; and two first pivotable links 48c, 48d which extend parallel to each other and each of which is pivotably connected, at two opposite ends thereof, to the first stationary link 48a and the first movable link 48b, respectively, so that the first stationary link 48a, the first movable link 48b, and the two first pivotable links 48c, 48d cooperate with each other to define a parallelogram. The first stationary link 48a is fixed to the rotatable member 46 such that the first movable link 48b is movable in a plane containing the vertical axis line C. In association with the first link device 48, there is provided a first coil spring 49 functioning as a first biasing device that produces a thrust having a directional component resisting a load applied to the first movable link 48b. The first coil spring 49 is connected at one end thereof to a connection point where one first pivotable link 48c and the first stationary link 48a are connected to each other, and is connected at the other end thereof to a connection point where the other first pivotable link 48d and the first movable link 48b are connected to each other, such that a moment produced by the first coil spring 49 in a direction to move the first movable link 48b upward, and a moment produced by the load applied to the first movable link 48b in a direction to move the same 48b downward are substantially cancelled by each other.

The second link device 50 includes a pair of second pivotable links 50c, 50d that extend parallel to each other; and the second stationary link 50a and a second movable link 50b which extend parallel to each other and each of which is pivotably connected, at two opposite ends thereof, to the two second pivotable links 50c, 50d, respectively, so that the second stationary link 50a, the second movable link 50b, and the two second pivotable links 50c, 50d cooperate with each other to define a parallelogram. The second stationary link 50a is fixed to the first movable link 48b such that the second stationary link 50a extends in a direction substantially perpendicular to the first stationary link 48a, and such that the second movable link 50b is movable in the plane containing the vertical axis line C. In association with the second link device 50, there is provided a second coil spring 51 functioning as a second biasing device that produces a thrust having a directional component resisting a load applied to the second movable link 50b. The second coil spring 51 is connected at one end thereof to a connection point where one second pivotable link 50c and the second stationary link 50a are connected to each other, and is connected at the other end thereof to a connection point where the other second pivotable link 50d and the second movable link 50b are connected to each other, such that a moment produced by the second coil spring 51 in a direction to move the second movable link 50b upward, and a moment produced by the load applied to the second movable link 50b in a direction to move the same 50b downward are substantially cancelled by each other. Owing to the respective moment-canceling actions of the first and second coil springs 49, 51, the sensor holding apparatus 10 can hold the ultrasonic probe 12 such that the probe 12 is stopped at a desirable position, or is slowly moved downward, in the three-dimensional space, and such that the outer surface of the free end portion 24 of the probe 12 lightly touches the skin surface 18 without deforming the blood vessel 20 and closely contacts the same 18 via the coupling agent such as the jelly.

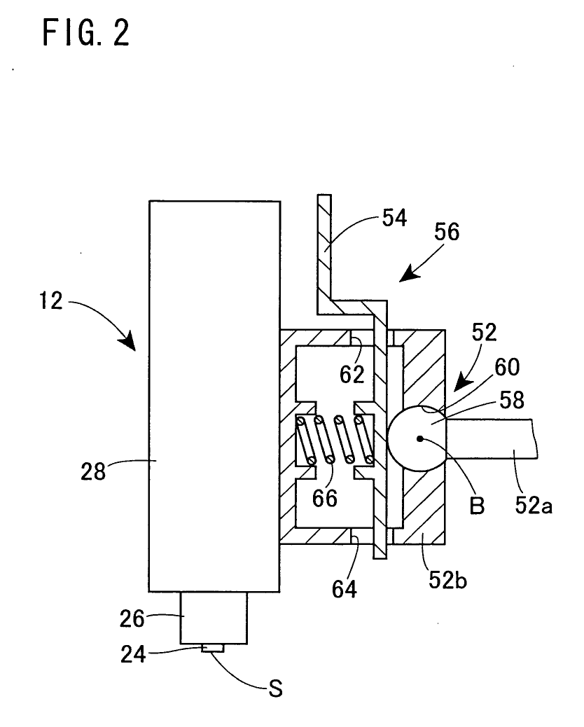

FIG. 2 is an enlarged view of the universal joint 52 and the stopper device 56. As shown in the figure, the universal joint 52 includes a first connection member 52a having a base end portion fixed to the second movable link 50b, and a free end portion 58 having a spherical shape; and a second connection member 52b that has a fitting hole 60 in which the spherical end portion 58 of the first connection member 52a slideably fits, and that is connected to the spherical end portion 58 such that the second connection member 52b is universally rotatable about a center, B, of the spherical portion 58. The second connection member 52b has two guide holes 62, 64 that cooperate with each other to guide the operable lever 54 of the stopper device 56 such that the operable lever 54 is movable toward, and away from, the spherical end portion 58 of the first connection member 52a.

The stopper device 56 includes, in addition to the operable lever 54, a pressing spring 66 that presses the operable lever 54 against the spherical end portion 58 of the first connection member 52a. In a usual state in which the operable lever 54 is not in use, the pressing spring 66 presses the operable lever 54 against the spherical portion 58, so as to inhibit the rotation of the universal joint 52 and thereby fix the same 52. However, when the operable lever 54 is used or operated by the operator against the biasing force of the pressing spring 66, and is moved away from the spherical portion 58, the fixation of the universal joint 52 is released and the universal rotation of the same 52 is permitted. Thus, the ultrasonic probe 12 can take a desirable posture.

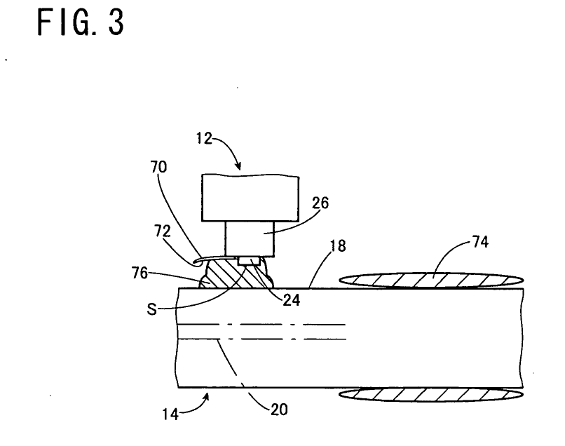

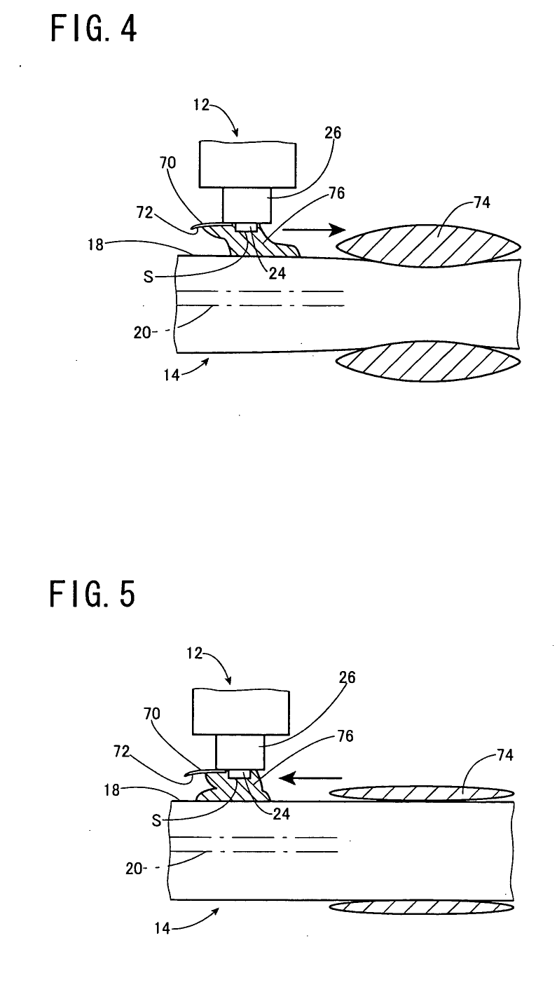

As shown in FIG. 3, the ultrasonic probe 12 has, in the free-end portion 24 as a lower-end portion thereof, a flange portion 70 that projects in a direction away from the emission surface S. The flange portion 70 is for interposing and keeping a coupling agent 76 such as a jelly between the emission surface S and the skin surface 18 even though the skin 18 may be moved during the inspection. Owing to the flange portion 70, a sufficiently large amount of the coupling agent 76 can be held by, and between, the skin surface 18 and a holding surface 72 of the flange portion 70 that is opposed to the skin surface 18. When the function of endothelium of the blood vessel 20 is evaluated by measuring a rate (%) of change of diameter of the blood vessel 20 that represents FMD (flow-mediated dilation) following postischemia reactive hyperemia, first, flow of blood in the blood vessel 20 is stopped for an appropriate time duration by inflation of a cuff 74 that is wound around a forearm of the living being 14, then the stopping of the blood flow is ended by deflation of the cuff 74, and finally a change of diameter of the blood vessel 20 is calculated based on the short-axis images of the blood vessel 20 that are obtained by the ultrasonic probe 12. A state in which the above-indicated sufficient amount of the coupling agent 76 is present between the emission surface S and the skin surface 18 means a state in which the coupling agent 76 is kept between the emission surface S and the skin surface 18 not only when the skin surface 18 is moved or drawn by the inflation of the cuff 70, as shown in FIG. 4, but also when subsequently the skin surface 18 is moved back by the deflation of the cuff 70, as shown in FIG. 5. In this state, the coupling agent 76 can transmit the ultrasonic waves with substantially no attenuation.

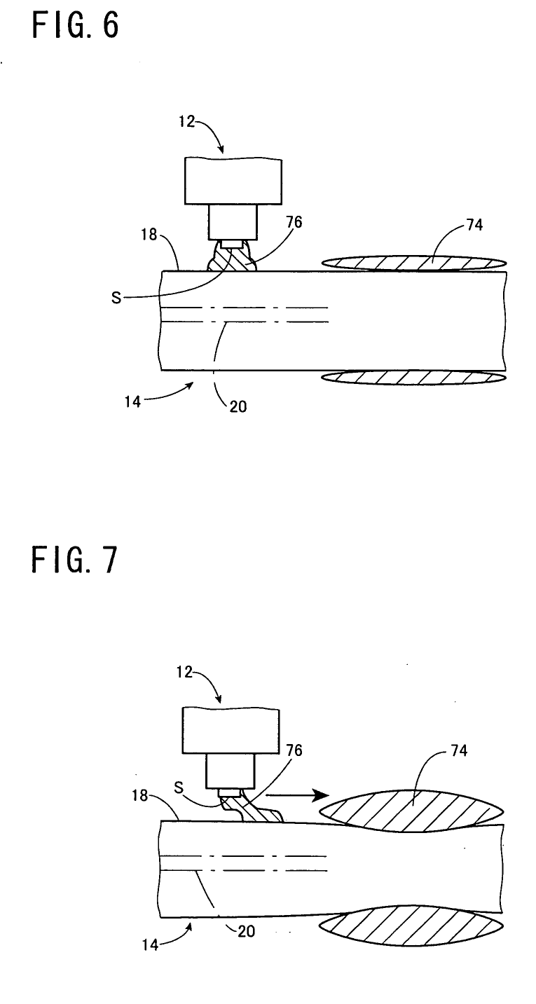

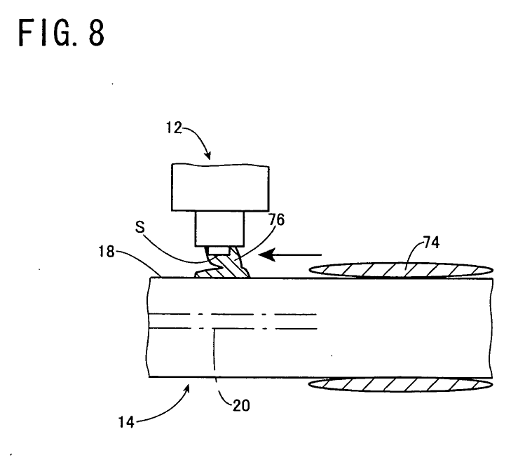

FIGS. 6, 7, and 8 show a case where the ultrasonic probe 12 does not employ the above-described flange portion 70. More specifically described, FIG. 6 shows a state before the blood vessel 20 is pressed by the cuff 74; FIG. 7 shows a state in which the skin surface 18 is moved or drawn by the inflation of the cuff 74; and FIG. 8 shows a state after the skin surface 18 is moved back by the deflation of the cuff 74. When the skin surface 18 is moved back, the coupling agent 76 present between the skin surface 18 and the emission surface S of the ultrasonic probe 12 is plastically deformed, and air is trapped in the path of transmission of the ultrasonic waves and causes a significant attenuation of the ultrasonic waves.

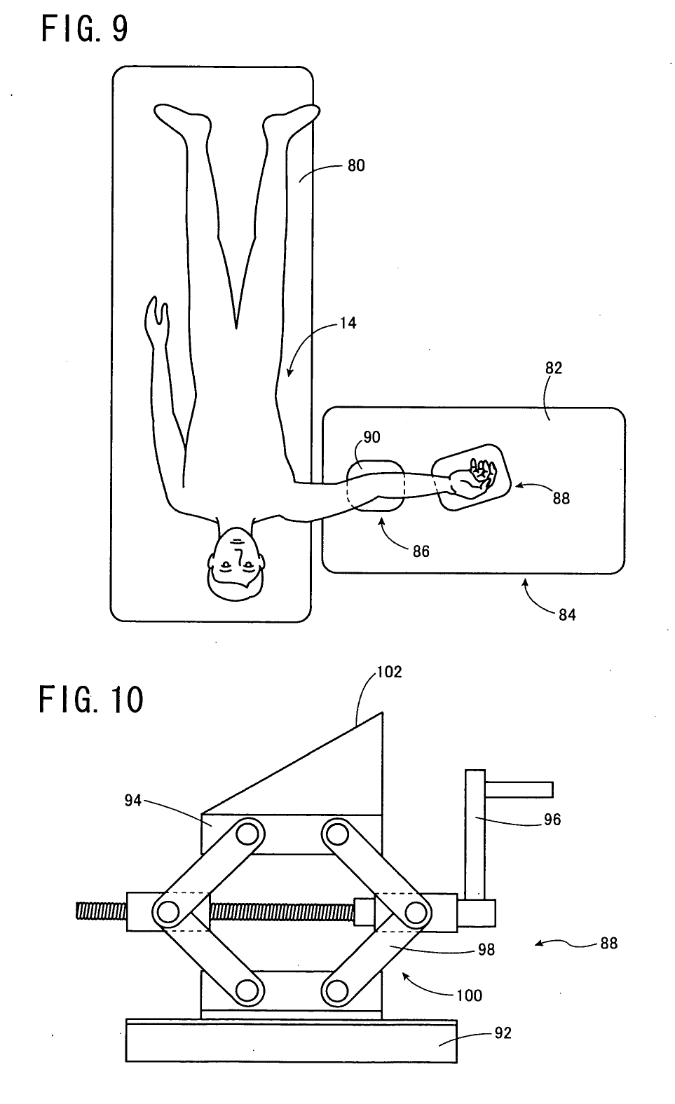

As shown in FIG. 9, the living being 14 as a living subject lies, on a bed 80, in a face-up position such that his or her arm is extended laterally. In order to measure, with accuracy, FMD following postischemia reactive hyperemia, it is needed to place the arm in a natural state, i.e., a relaxed state. To this end, in the present embodiment, a support table 84 having a horizontal support surface (i.e., top surface) 82 is employed, and an elbow-portion support member 86 that supports an elbow portion between the forearm and the upper arm of the living being 14, and a hand-dorsal-portion support member 88 that supports a dorsal portion of the corresponding hand of the living being 14 are placed on the support surface 82 of the support table 84 such that a position of each of the elbow-portion support member 86 and the hand-dorsal-portion support member 88 is changeable in horizontal directions. Each of the elbow-portion support member 86 and the hand-dorsal-portion support member 88 can be fixed to the support surface 82 of the support table 84 with, e.g., a magnetic attraction caused by a permanent magnet or an electromagnet, a vacuum suction produced in, or supplied to, the support surface 82, or a fastening force caused by a fastening member that extends in a through-hole formed through the thickness of the support surface 82.

The elbow-portion support member 86 is a block-like member having a substantially horizontal elbow-support surface 90. The hand-dorsal-portion support member 88 includes, as shown in FIG. 10, a base plate 92 and a support plate 94 that extend parallel to each other; and a height adjusting device 100 that includes a height adjusting handle 96 that can be manually rotated for adjusting a height of the support plate 94, and a pantagraph-like link device 98 that connects between the base plate 92 and the support plate 94. The support plate 94 has a support surface 102 that is inclined for the purpose of supporting the dorsal portion of the hand. Respective height positions of the respective support surfaces 90, 102 of the elbow-portion support member 86 and the hand-dorsal-portion support member 88 are so pre-selected as to be able to provide a sufficiently large space between the cuff 74 wound around the forearm and the support surface 82 of the support table 84. In addition, the height position of the support surface 102 of the hand-dorsal-portion support member 88 can be so adjusted as to be able to permit the living being 14 to relax his or her hand.

The present blood-vessel-endothelium-function inspecting apparatus 30 employs the elbow-portion support member 86 that supports the elbow portion between the forearm and the upper arm of the living being 14, and the hand-dorsal-portion support member 88 that supports the dorsal portion of the hand of the living being 14, and the two support members 86, 88 cooperate with each other to keep the cuff 74 wound around the forearm of the living being 14, away from the support surface 82 of the support table 84, during the inspection. Therefore, even if the cuff 74 may be inflated, the movement of the forearm can be effectively restrained. Thus, a relative movement of the blood vessel (i.e., brachial artery) 20 and the ultrasonic probe 12 supported by the sensor holding apparatus 10, that may be caused by the movement of the forearm in relation with the inflation of the cuff 74, can be advantageously prevented. That is, stable images of the blood vessel 20 can be obtained and diameters of the same 20 can be measured with accuracy based on the thus obtained images.

In addition, in the present blood-vessel-endothelium-function inspecting apparatus 30, the elbow-portion support member 86 and the hand-dorsal-portion support member 88 are placed on the horizontal support surface 82 of the support table 84, such that each of the two support members 86, 88 is movable in the horizontal directions. Therefore, the respective positions of the two support members 86, 88 can be changed to respective optimum positions corresponding to the conformation of the body of the living being 14 lying on the bed 80, so that the arm of the living subject 14 can take a natural or relaxed posture. Under this condition, the function of endothelium of blood vessel of the living being 14 can be inspected with reliability.

Moreover, in the present blood-vessel-endothelium-function inspecting apparatus 30, the hand-dorsal-portion support member 88 employs the height adjusting device 100 that can be manually operated to adjust the height position of the inclined support surface 102. Therefore, the height position of the inclined support surface 102 can be changed to an optimum position corresponding to the conformation of the body of the living being 14 lying on the bed 80, so that the arm of the living subject 14 can take a natural or relaxed posture. Under this condition, the function of endothelium of blood vessel of the living being 14 can be inspected with reliability.

In addition, in the present blood-vessel-endothelium-function inspecting apparatus 30, the ultrasonic probe 12 employs the holding surface 72 (or the flange portion 70) that is provided around the emission surface S so as to be opposed to the skin surface 18 and that holds the coupling agent 76 between the emission surface S and the skin surface 18 even if the skin surface 18 may be moved by the inflation of the cuff 74 during the inspection. That is, even if the cuff 74 may be inflated and accordingly the skin surface 18 may be moved or drawn, the coupling agent 76 such as the jelly can be held or kept between the emission surface S and the skin surface 18. Thus, stable images of the blood vessel 20 can be obtained and diameters of the same 20 can be measured with accuracy based on the thus obtained images.

In addition, in the present blood-vessel-endothelium-function inspecting apparatus 30, the ultrasonic probe 12 employs the flange portion 70 that is formed of a metal plate or a resin plate and that projects in the direction away from the emission surface S of the free-end portion 24 and away from the cuff 74. The flange portion 70 has the above-described holding surface 72 such that the holding surface 72 is opposed to the skin surface 18. Thus, even if the skin surface 18 may be moved or drawn because of the inflation of the cuff 74, the coupling agent 76 such as the jelly can be reliably held or kept between the emission surface S and the skin surface 18. Therefore, stable images of the blood vessel 20 can be obtained and diameters of the same 20 can be measured with accuracy based on the thus obtained images.

Hereinafter, there will be described other embodiments of the present invention. In the following description, the same reference numerals as used in the first embodiment shown in FIGS. 1 through 10 are used to designate the corresponding elements or parts of the other embodiments and the description thereof is omitted.

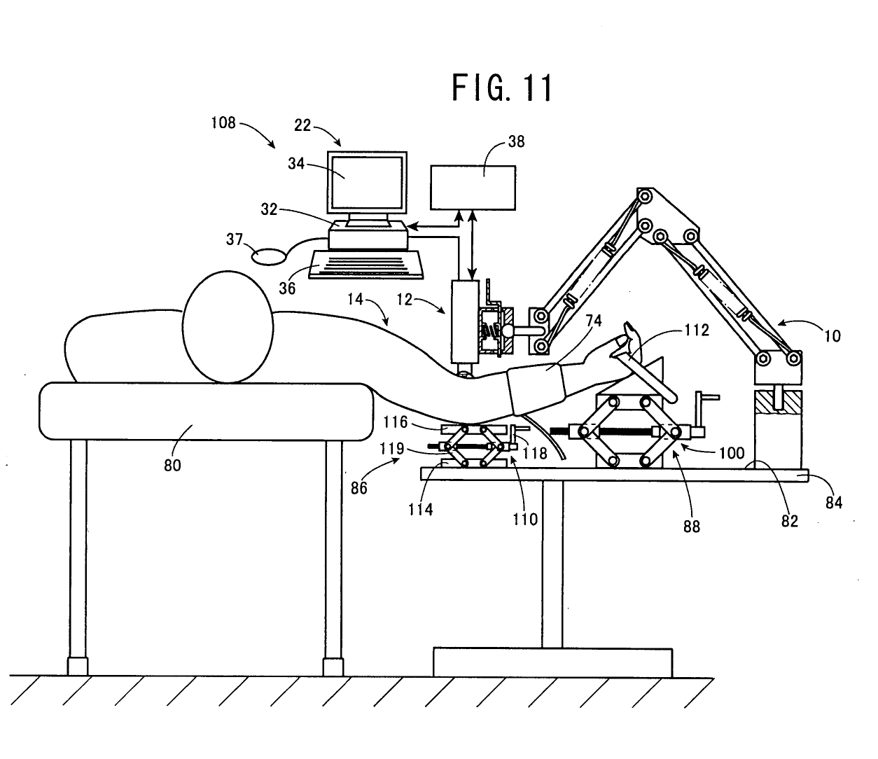

FIG. 11 shows another blood-vessel-endothelium-function inspecting apparatus 108 as a second embodiment of the present invention. The present blood-vessel-endothelium-function inspecting apparatus 108 has a construction basically identical with that of the blood-vessel-endothelium-function inspecting apparatus 30 as the first embodiment, and differs from the apparatus 30 only in that the apparatus 108 includes an elbow-portion support member 86 that is not a block-like member but employs a height adjusting device 110 similar to the height adjusting device 100 of the hand-dorsal-portion support member 88 of the apparatus 30; and a hand-dorsal-portion support member 88 that employs, in addition to a height adjusting device 100, an annular belt 112 that is grasped by a hand of a living being 14 when a dorsal portion of the hand is supported by the support member 88.

As shown in FIG. 11, the elbow-portion support member 88 includes a base plate 114 and a support plate 116 that extend parallel to each other; and the height adjusting device 110 that includes a height adjusting handle 118 that can be manually rotated for adjusting a height of the support plate 114, and a pantagraph-like link device 119 that connects between the base plate 114 and the support plate 116. In addition, the hand-dorsal-portion support member 88 includes the annular belt 112 that is grasped by the hand of the living being 14 when the dorsal portion of the hand is supported by an inclined support surface 102 of the support member 88.

In the present blood-vessel-endothelium-function inspecting apparatus 108, the elbow-portion support member 86 employs the height adjusting device 110 similar to the height adjusting device 100 of the hand-dorsal-portion support member 88. Therefore, a height of the elbow-portion support member 86 can be easily changed to an optimum value corresponding to the conformation of the body of the living being 14 lying on a bed 80, so that the arm of the living subject 14 can take a natural or relaxed posture. Under this condition, the function of endothelium of blood vessel of the living being 14 can be inspected with reliability.

Moreover, in the present blood-vessel-endothelium-function inspecting apparatus 108, the hand-dorsal-portion support member 88 employs the annular belt 112 that is grasped by the hand of the living being 14 when the dorsal portion of the hand is supported by the inclined support surface 102. Therefore, the hand and arm of the living being 14 can be held with stability during the inspection. Thus, stable images of a blood vessel 20 can be obtained and diameters of the same 20 can be measured with accuracy based on the thus obtained images.

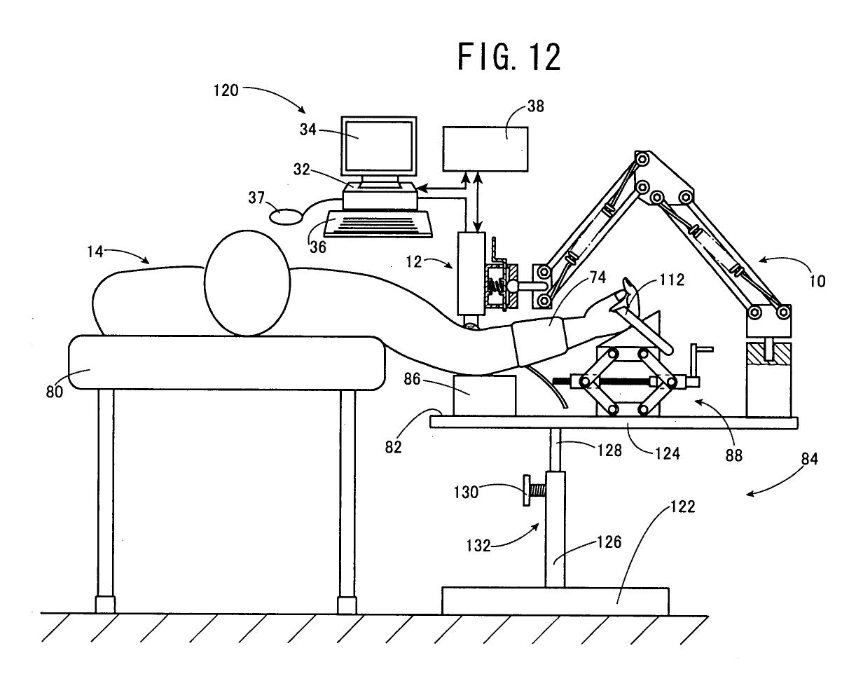

FIG. 12 shows another blood-vessel-endothelium-function inspecting apparatus 120 as a third embodiment of the present invention. The present blood-vessel-endothelium-function inspecting apparatus 120 has a construction basically identical with that of the blood-vessel-endothelium-function inspecting apparatus 30 as the first embodiment, and differs from the apparatus 30 only in that the apparatus 120 employs a support table 84 whose horizontal support surface 82 supports an elbow-portion support member 86 and a hand-dorsal-portion support member 88, and whose height can be adjusted, and in that the hand-dorsal-portion support member 88 employs an annular belt 112 that is grasped by a hand of a living being 14 when a dorsal portion of the hand is supported by the support member 88.

As shown in FIG. 12, the support table 84 includes a base plate 122; a top plate 124 whose upper surface functions as the horizontal support surface 82; and an extensible column 132 including a tubular member 126 that projects upward from the base plate 122, an axial member 128 that is fixed to a lower surface of the top plate 124 and is inserted in the tubular member 126, and a locking screw 130 that is threadedly engaged with the tubular member 126 so as to fix the axial member 128 to the tubular member 126 at a desired position. Thus, a total length of the column 132 can be changed and accordingly a height position of the top plate 124 can be adjusted.

In the present blood-vessel-endothelium-function inspecting apparatus 120, the support table 84 whose horizontal support surface 82 supports the elbow-portion support member 86 and the hand-dorsal-portion support member 88, is constructed such that the height of the support table 84 can be adjusted. Therefore, respective heights of the elbow-portion support member 86 and the hand-dorsal-portion support member 88 can be easily changed to respective optimum values corresponding to the conformation of the body of the living being 14 lying on a bed 80, so that the arm of the living subject 14 can take a natural or relaxed posture. Under this condition, the function of endothelium of blood vessel of the living being 14 can be inspected with reliability.

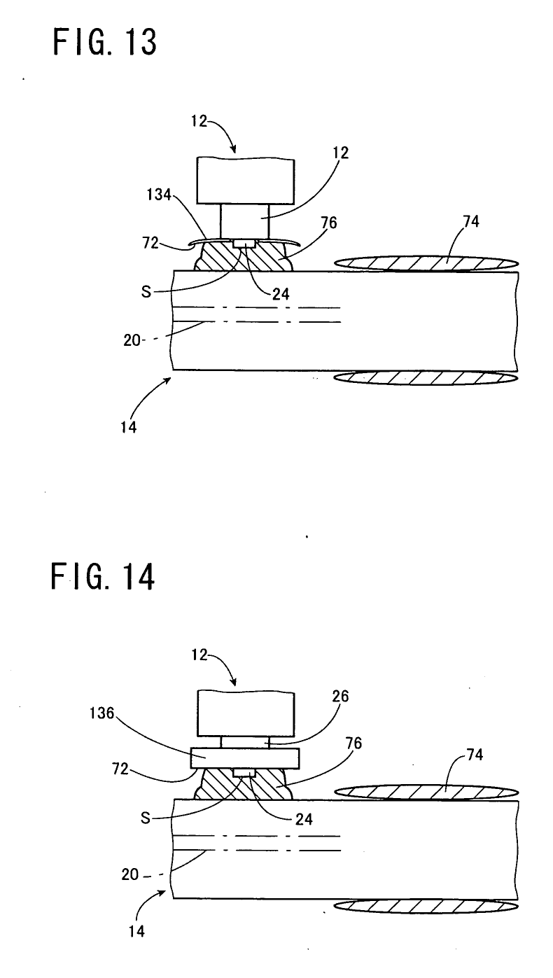

FIGS. 13 and 14 show respective modified embodiments of a holding surface 72 that is provided in the free-end portion 24 of the ultrasonic probe 12 so as to hold the coupling agent 76. In the first embodiment shown in FIGS. 3 through 5, the flange portion 70 has a shape that projects from the free-end portion 24 or the emission surface S in a direction away from the cuff 74. In contrast, in each of the modified embodiments shown in FIGS. 13 and 14, a circular or annular holding surface 72 is provided around the emission surface S provided at the center of the lower end surface of the free-end portion 24, such that a center of the holding surface 72 rides on the emission surface S. More specifically described, in the modified embodiment shown in FIG. 13, a flange portion 134 that is formed of a circular metal or resin plate is provided in the free-end portion 24 of the ultrasonic probe 12 such that the flange portion 134 projects in directions away from the emission surface S. The flange portion 134 has, as a surface thereof opposed to the skin surface 18, a circular holding surface 72 whose center rides on the emission surface S. In addition, in the modified embodiment shown in FIG. 14, a circular large-diameter portion 136 is provided around the free-end portion 24 of the ultrasonic probe 12, and has, as a surface thereof opposed to the skin surface 18, a circular holding surface 72 whose center rides on the emission surface S. In each of the modified embodiments shown in FIGS. 13 and 14, a sufficiently large amount of coupling agent 76 can be provided between the skin surface 18 and the circular holding surface 72 whose center rides on the emission surface S. Therefore, not only when the skin surface 18 is moved or drawn by the inflation of the cuff 74 to stop the flow of blood in the blood vessel 20, but also when subsequently the skin surface 18 is moved back to its initial position by the deflation of the cuff 74 to end the stopping of blood flow, the coupling agent 76 such as the jelly can remain with reliability between the skin surface 18 and the emission surface S of the ultrasonic probe 12. Thus, stable images of the blood vessel 20 can be obtained and diameters of the same 20 can be measured with accuracy based on the thus obtained images.

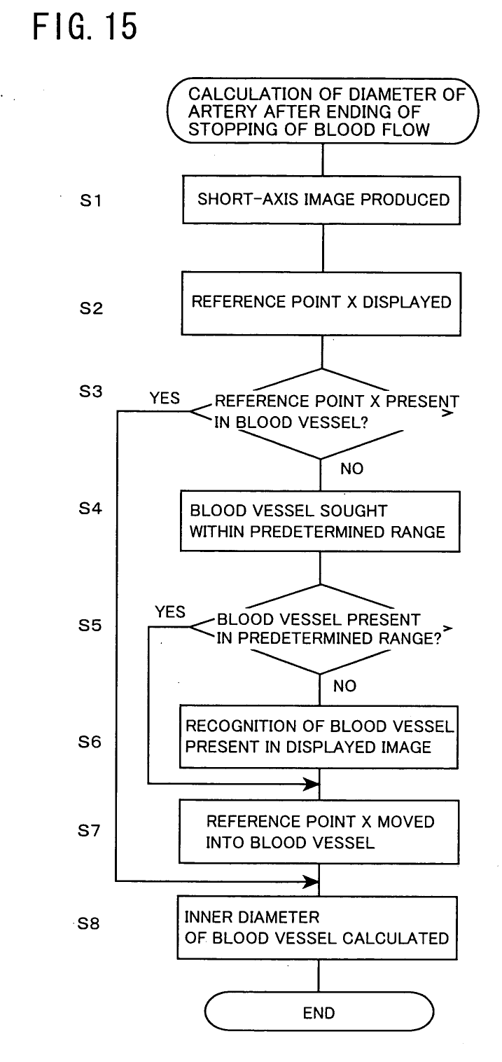

In each of the above-described embodiments shown in FIGS. 1 through 14, the electronic control device 32 operates for measuring or calculating, according to a control program represented by a flow chart shown in FIG. 15, a diameter of an endothelium (i.e., an inner layer) of a blood vessel (i.e., an artery) 20 after stopping of blood flow is ended. According to the flow chart, the control device 32 operates for seeking the blood vessel 20 in a transverse-cross-section image, i.e., a short-axis image thereof, as described below.

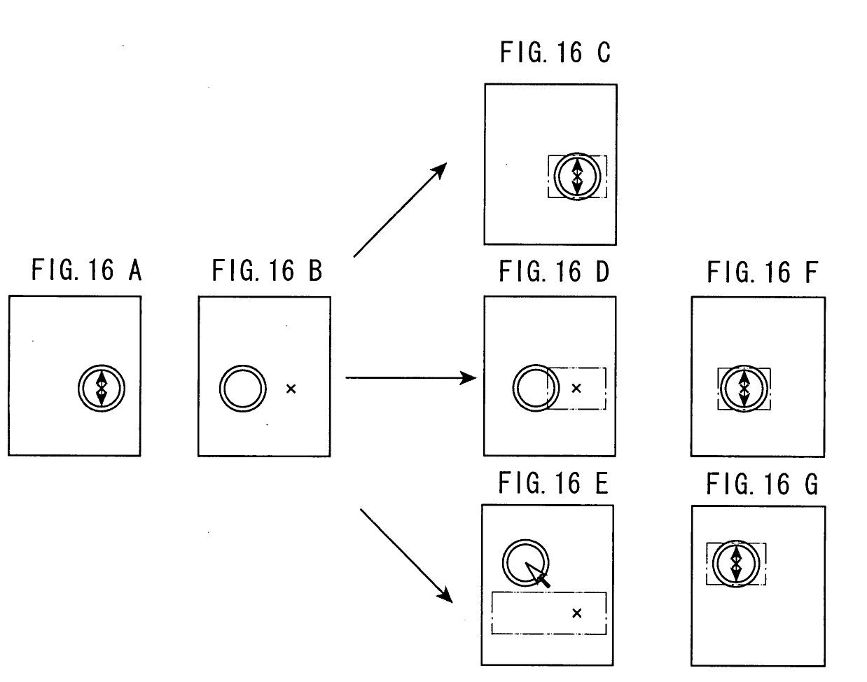

First, at Step S1, the control device 32 produces, based on the ultrasonic waves received by the ultrasonic probe 12 (i.e., the ultrasonic waves reflected from the blood vessel 20), a short-axis image of the blood vessel 20. In the present embodiment, Step S1 corresponds to a cross-section-image producing means or device. FIG. 16A shows a short-axis image of the blood vessel 20 before the flow of blood in the blood vessel 20 is stopped by the inflation of the cuff 74. A reference point, X, representing a position of the image of the blood vessel 20 shown in FIG. 16A is stored by the control device 32. FIG. 16B shows a short-axis image of the blood vessel 20 when the flow of blood in the blood vessel 20 is being stopped by the inflation of the cuff 74. Since the skin surface 18 is drawn by the inflation of the cuff 74, the image of the blood vessel 20 shown in FIG. 16B is deviated from the reference point X. FIGS. 16C, 16D, and 16E show different examples of the short-axis image of the blood vessel 20 produced at Step S1.

At Step S2, the control device 32 controls the monitor-image displaying device 34 to display, with the produced short-axis image of the blood vessel 20, the reference point X representing the position of the image of the blood vessel 20 before the flow of blood in the blood vessel 20 is stopped by the inflation of the cuff 74. Each of FIGS. 16C, 16D, and 16E shows this state. Next, at Step S3, the control device 32 judges whether the reference point X is present in the short-axis image of the blood vessel 20 displayed at Step S2. Regarding the example shown in FIG. 16C, a positive judgment is made at Step S3 and the control of the control device 32 goes to Step S8 to calculate, according to a pre-stored blood-vessel-inner-diameter calculation algorithm, a diameter of the inner layer (i.e., tunica intima) of the blood vessel 20 surrounding the reference point X. In the present embodiment, Step S8 corresponds to a blood-vessel-diameter calculating means or device.

On the other hand, regarding the example shown in FIG. 16D or FIG. 16E, a negative judgment is made at Step S3, and the control goes to Step S4 to seek the image of the blood vessel 20 within a predetermined image-seek range, indicated by one-dot chain line, that corresponds to the reference point X. The predetermined image-seek range is such a range that presence of images of veins and/or tendons cannot be expected but presence of the artery 20 only can be expected. In the present embodiment, Step S4 corresponds to a blood-vessel-position seeking means or device.

Then, at Step S5, the control device 32 judges whether the image of the blood vessel 20 has been found within the predetermined image-seek range. Regarding the example shown in FIG. 16D, a positive judgment is made at Step S5, because the reference point X is positioned in the vicinity of the short-axis image of the blood vessel 20 displayed at Step S2. In this case, the control goes to Step S7 to move the reference point X to a position in the blood vessel 20 whose image has been found, as shown in FIG. 16F. Step S7 is followed by Step S8. On the other hand, regarding the example shown in FIG. 16E, a negative judgment is made at Step S5, because the reference point X is remote from the short-axis image of the blood vessel 20 displayed at Step S2. In this case, the control goes to Step S6 where an operator seeks the blood vessel 20 whose cross-section image is displayed by the displaying device 34. In addition, the operator operates the mouse 37 as the manually operable input device to move the reference point X to a position inside the blood vessel 20 displayed by the displaying device 34, as shown in FIG. 16G. Then, the control goes to Step S7.

Thus, the electronic control device 32 includes (a) the cross-section-mage producing means or device S1 that produces the cross-section image of the blood vessel 20 based on the reflected ultrasonic waves detected by the ultrasonic probe 12; (b) the blood-vessel-position seeking means or device S4 that seeks, in the cross-section image obtained after stopping of the flow of blood is ended, the image of the blood vessel 20 within the predetermined image-seek range corresponding to the reference point X representing the position of the blood vessel 20 before the flow of blood is stopped; and (c) the blood-vessel-diameter calculating means or device S8 that calculates the diameter of the image of the blood vessel 20 found by the blood-vessel-position seeking means or device S4. Therefore, even if the image of the blood vessel 20 may be moved because the skin surface 18 is drawn by the inflation of the cuff 74 and/or the forearm is moved, the blood-vessel-position seeking means or device S4 seeks the image of the blood vessel 20 and the blood-vessel-diameter calculating means or device S8 calculates the diameter, d, of the sought and found image of the blood vessel 20. Thus, the diameter of the blood vessel 20 can be measured with reliability.

While the present invention has been described in its preferred embodiments by reference to the drawings, it is to be understood that the invention may otherwise be embodied.

For example, in each of the first embodiment shown in FIG. 1 and the third embodiment shown in FIG. 12, the elbow-portion support member 86 whose height is unchangeable and the hand-dorsal-portion support member 88 whose height is changeable are employed; and in the second embodiment shown in FIG. 11, the elbow-portion support member 86 whose height is changeable and the hand-dorsal-portion support member 88 whose height is changeable are employed. However, it is possible to employ the elbow-portion support member 86 whose height is unchangeable and the hand-dorsal-portion support member 88 whose height is unchangeable, or employ the elbow-portion support member 86 whose height is changeable and the hand-dorsal-portion support member 88 whose height is unchangeable.

In addition, in each of the first, second, and third embodiments respectively shown in FIGS. 1, 11, and 12, the elbow-portion support member 86 and the hand-dorsal-portion support member 88 are provided on the support surface 82 of the support table 84, such that each of the two members 86, 88 is movable. However, it is possible to provide the elbow-portion support member 86 and the hand-dorsal-portion support member 88 on the support surface 82 of the support table 84, such that one of the two members 86, 88 is movable and the other member is fixed in position.

In addition, in each of the first, second, and third embodiments respectively shown in FIGS. 1, 11, and 12, the elbow-portion support member 86 is adapted to support the elbow portion between the forearm, and the upper arm, of the living being 14. However, the elbow-portion support member 86 may be adapted to support a portion of the forearm that is adjacent the elbow portion, or a portion of the upper arm that is adjacent the elbow portion.

In addition, in the first embodiment shown in FIG. 1, the sensor holding apparatus 10 including the two link devices 48, 50 is employed to hold the ultrasonic probe 12. However, it is possible to employ a different sort of sensor holding apparatus, such as a slide arm or a robot arm.

In addition, in each of the illustrated embodiments, the height adjusting device 100, 110 is constituted by the pantagraph-type link device. However, it is possible to employ a different sort of height adjusting device including, e.g., an extensible (or retractable) rod.

In addition, in each of the first, second, and third embodiments respectively shown in FIGS. 1, 11, and 12, the living being 14 lies, during the inspection, in the face-up position on the bed 80. However, the living being 14 may sit, during the inspection, in a seat in a relaxed state.

The present invention may be embodied with various changes and improvements that may occur to a person skilled in the art, without departing from the spirit and scope of the invention.

What is claimed is:

1. An apparatus for inspecting a function of an endothelium of a blood vessel of a living being, by stopping, using an inflatable cuff wound around a forearm of the living being, a flow of blood in a blood vessel of the forearm, subsequently ending the stopping of the flow of blood, and causing an ultrasonic probe placed on a skin of an upper arm of the living being to emit, from an emission surface thereof, an ultrasonic wave toward a blood vessel under the skin, the apparatus comprising:
an elbow-portion support member which supports an elbow portion of the living being; and
a hand-dorsal-portion support member which supports a dorsal portion of a hand of the living being and which cooperates with the elbow-portion support member to keep, in a space, the inflatable cuff wound around the forearm of the living being.

2. The apparatus according to claim 1, wherein at least one of the elbow-portion support member and the hand-dorsal-portion support member is provided on a horizontal support surface of a support table such that said at least one of the elbow-portion support member and the hand-dorsal-portion support member is movable in a horizontal direction on the horizontal support surface.

3. The apparatus according to claim 1, further comprising a height adjusting device which adjusts a height of a support surface of at least one of the elbow-portion support member and the hand-dorsal-portion support member that supports a corresponding one of the elbow portion, and the dorsal portion of the hand, of the living being.

4. The apparatus according to claim 1, further comprising a belt which is attached to the hand-dorsal-portion support member and which is to be grasped by the hand of the living being when the dorsal portion of the hand is supported by the hand-dorsal-portion support member.

5. An apparatus for inspecting a function of an endothelium of a blood vessel of a living being, by causing an ultrasonic probe placed on a skin of a portion of a living being to emit, from an emission surface thereof, an ultrasonic wave toward a blood vessel under the skin, the apparatus comprising:
a holding surface which is provided in a vicinity of the emission surface of the ultrasonic probe such that the holding surface is opposed to the skin, so that a coupling agent is interposed between the emission surface and the skin irrespective of whether the skin may be moved during the inspection.

6. The apparatus according to claim 5, wherein the ultrasonic probe includes an end portion which supports a flange portion projecting therefrom in a direction away from the emission surface, and the flange portion has the holding surface as a surface thereof that is opposed to the skin.

7. An apparatus for inspecting a function of an endothelium of a blood vessel of a living being, by causing an ultrasonic probe placed on a skin of a portion of a living being to emit, from an emission surface thereof, an ultrasonic wave toward a blood vessel under the skin, the apparatus comprising:
a cross-section-image producing device which produces, based on the reflected ultrasonic wave detected by the ultrasonic probe, a cross-section image of the blood vessel;
a blood-vessel-position seeking device which seeks, in a cross-section image of the blood vessel produced after stopping of a flow of blood in a blood vessel is ended, the blood vessel within a pre-set range corresponding to a position of the blood vessel before the flow of blood is stopped; and
a blood-vessel-diameter calculating device which calculates a diameter of the blood vessel sought by the blood-vessel-position seeking device.
